(19) United States
(12) Patent Application Publication
(10) Pub. No.: US 2004/0066199 A1
(43) Pub. Date: Apr. 8, 2004

(54) Evolutionary algorithm for modeling ion channels

(76) Inventor: Paul A. Rhodes, West Palm Beach, FL (US)

Correspondence Address: FISH & RICHARDSON P.C., 1425 K STREET, N.W., 11TH FLOOR, WASHINGTON, DC, 20005-3500, US

(21) Appl. No.: 10/455,784

(22) Filed: Jun. 6, 2003

Related U.S. Application Data
(60) Provisional application No. 60/385,910, filed on Jun. 6, 2002.

(51) Int. Cl. G01N 27/26; C12M 1/00

(52) U.S. Cl. 324/500; 204/403.06

(57) ABSTRACT

A method of using experimental data determines the structure and voltage dependence of transition rates for states in models of ion channels.

### CLAIM OF PRIORITY

This application claims priority under 35 USC §119(e) to U.S. patent application Ser. No. 60/385,910, filed on Jun. 6, 2002, the entire contents of which are hereby incorporated by reference.

### TECHNICAL FIELD

This invention relates to an evolutionary algorithm for modeling ion channels, and more particularly to an automatic procedure to develop models of ion channels.

### BACKGROUND

Ion channels play important roles in living systems, ranging from the simplest algae to the mammalian brain. Ion channels allow current to flow across cell membrane separating the inner and outer ionic milieu, usually a selective opening passing a limited subset of ionic species, and generally a gated opening, often occurring as a specific function of transmembrane voltage, intracellular calcium ion concentration, or local binding of GTP-activated proteins. See Hille, B., (2001) Ion Channels of Excitable Membranes, 3^{rd} Edition, Sinauer: Sunderland, Mass., which is hereby incorporated by reference in its entirety. The functions served by ion channels range from controlling osmotic pressure in simple single celled organisms to the subtle regulation of integration of inputs in brain circuits. The ancient nature of the role played by ion channels is apparent in the similarity between the channel families found in algae, earthworms, and humans. Many of the main channel families present in humans, including several classes of voltage-gated potassium and calcium ion channels, are elaborations of the smaller set of channels found in Drosophila, molecular ancestors of which arose in algae. As indication of the centrality of ion channels in the mechanisms of life, consider that 35% of the proteins coded by the C. elegans genome are believed to code for ion channels or for enzymes which specifically modulate ion channels. At the beginning of 2002, some 200 distinct ion channels had been identified in mammals. Potassium channels alone form 13 identified families including 130 distinct channels. See Rudy, B., (1999) Annals of the New York Academy of Science 868:1-12, which is hereby incorporated by reference in its entirety. This number is a remarkable 5-fold greater than 5 years ago, and is likely to rise further as the genomes of humans and many other species are decoded and interpreted. The sheer vastness of the ion channel palette suggests the absolutely central role they play in the functions of life.

Furthermore, the kinetic properties of each of these distinct channels and variants is modified by perhaps 20 or more known enzymes, generally kinases and phosphatases. See Catterall, W A (1997), Advances in Second Messenger and Phosphoprotein Research 31:159-181, which is hereby incorporated by reference in its entirety. Neuronal, cardiac, and other physiological functions are well known to be controlled by an extraordinarily subtle and rich interaction of nonlinear (voltage-gated) currents, often with many distinct currents present in the same membrane. The prospect of dynamic interactions among these currents suggests that biological systems employ a dauntingly complex regime of functional control by the selective regulation of a host of nonlinearly interacting ion channels.

Despite their central role in life, there was no physical model of ion channel function until the 1950's when Alan Hodgkin and Andrew Huxley's Nobel prize-winning studies of the sodium and potassium ion conductances in the squid giant axon led to their development of kinetic models which allowed the detailed account of the properties of action potential generation and conduction. See Hodgkin, A L, Huxley A F (1952) Journal of Physiology, 117: 500-544, which is hereby incorporated by reference in its entirety. Thus they developed the first models of the kinetics of voltage-gated membrane conductances, work which enabled a comprehensive understanding of the digital transmission of signals in the nervous system. At this time (1952) the existence of channels themselves was uncorroborated and their actual structure completely unknown; measurement was limited to macroscopic permeabilities in membrane. Though the Hodgkin-Huxley (HH) model form, positing physically independent processes of channel opening (activation) and self-shutting (inactivation) has proven to be biophysically inaccurate, models of this class have provided a remarkably close approximation to channel function, and are the cornerstone of most neuronal and cardiac modeling throughout both neuroscience and theoretical cardiology. HH models of ion channels have been and continue to be of immense value.

The methodology most commonly used today to develop mathematical models of ion channels is based on the Hodgkin Huxley model. The HH model has two salient weaknesses. First, the HH model relies upon an admittedly nonphysical model, in which the processes of activation and inactivation of channel conductance are artificially separated. An alternative and more biophysically accurate ion channel modeling framework, the Markov state model, exists, in which a set of closed, open and inactivated states are all mutually interlinked with rates of transition between each state pair dependent upon voltage. It is thought that the state transitions map to allosteric shifts in configuration of the proteins which make up physical ion channels, and thus Markov state channel models have great generality, power, and physical relevance; however, it can be notoriously laborious to determine the optimum number closed, open and inactivated states given channel data and the parameterization of the voltage-dependent transitions between them.

Second, the Hodgkin Huxley model and the more powerful Markov state channel model both rely upon a series of measurements at a set of fixed, or clamped, membrane voltages. The clamped membrane voltage was an innovation introduced by Cole and Curtis and perfected by Hodgkin and Huxley to remove complicating effects of time-varying voltage to simplify parameter determination. See Cole, K. S., and Curtis, H. J. (1939) Journal of General Physiology, 22: 649-670, which is hereby incorporated by reference in its entirety. One limitation of this methodology, which heretofore has not been addressed, is that model parameters are extracted from observations at fixed voltages, while real channels in biological conditions operate in the context of rapidly changing membrane voltage levels, which rise at the rate of up to 500 V/sec during the action potentials which are the hallmark of neuronal and cardiac processes.

### SUMMARY

In general, a model of an ion channel is sought by an evolutionary algorithm. The evolutionary algorithm selects from a population those models that can more accurately simulate experimental results.

In one aspect, a multistate model for an ion channel includes a plurality of states, each state capable of a transition to each other state, each transition being described by a plurality of parameters, wherein the parameters are adjusted via an evolutionary algorithm. Each transition can be a voltage-dependent transition. The model can include two or more states. The states can include an open state, a closed state, and optionally one or more inactivated states. The model can include three or more states. When the model includes three or more states, the states can include an open state, a closed state, and an inactivated state. Each transition can be described by at least three parameters. The parameters can include a rate parameter, a voltage equilibrium parameter, and a charge parameter.

In another aspect, a method of modeling ion channel behavior includes simulating a result with a multistate ion channel model having two or more states, comparing the simulated result to an experimental result to provide a measure of fitness, and altering the multistate ion channel model based on the measure of fitness. The multistate ion channel model can include a plurality of states, each state capable of a transition to each other state, each transition being described by a plurality of parameters. Each transition can be a voltage-dependent transition. The multistate ion channel model can be a member of a population of multistate ion channel models. The method can include selecting a member of the population after comparing each simulated result to an experimental result. Simulating the result and comparing the simulated result to an experimental result can be performed in parallel on each member of the population.

The states can include an open state, a closed state, and optionally one or more inactivated states. Each transition can be described by at least three parameters. The parameters can include a rate parameter, a voltage equilibrium parameter, and a charge parameter. The method can be iterated until the measure of fitness reaches a predetermined level of fitness. Altering the multistate ion channel model can include swapping a parameter of a first member of the population with a parameter of a second member of the population. Altering the multistate ion channel model can include altering the number of states. The experimental result can include an electrical measurement of a cell. The experimental result can include an electrical measurement of at least a portion of a cell membrane. Comparing the simulated result to the experimental result to provide a measure of fitness can include ranking the members of the population by the measure of fitness. Simulating a result can include predicting ion channel behavior in the presence of a modulator. The experimental result can include an electrical measurement of a cell measured in the presence of a modulator. The experimental result can include an electrical measurement of at least a portion of a cell membrane measured in the presence of a modulator. The multistate ion channel model can be a model for a sodium channel, a potassium channel, a calcium channel, or a combination thereof.

In another aspect, a system for modeling ion channel behavior includes a data input device configured to provide electrical recordings of a cell or a cell membrane, a data analysis device electrically connected to the data input device, and an output device electrically connected to the data analysis device. The data input device can include an electrode for recording an electrical signal of a cell or a cell membrane. The data input device can include a stored library of electrical signals recorded from a cell or a cell membrane. The stored library of electrical signals recorded from a cell membrane can include a signal recorded in the presence of an ion channel modulator. The library of electrical signals recorded from a cell membrane can include a signal recorded after a train of electrical pulses was applied to the cell membrane. The data analysis device can compute a mathematical simulation of an electrical recording of a cell or a cell membrane. The data analysis device can compare the mathematical simulation to an experimental recording of a cell or a cell membrane.

The measure of fitness can be the accuracy of a simulation compared against comparable experimental ion currents. The experimental ion currents can be from, for example, a cell, an oocyte or other expression system, an isolated real or artificial membrane, or a portion of a membrane, as with a patch electrode measurement. It not necessary to know the initial channel density in the membrane a priori. The method can accommodate the presence of more than a single channel type without knowing the relative proportions of types.

Both Hodgkin Huxley and Markov state model parameters can be determined by utilizing an evolutionary search of parameter space. Advantageously, this methodology lends itself to the systematic automation of ion channel model determination. An automated approach may be needed to explore the effects of candidate drugs upon the hundreds of ion channel types and subtypes which recent research has revealed underlie all physiological function. Further, as detailed below, the methodology can be used with measured ion channel currents occurring during arbitrarily complex voltage patterns, such as the trains of spikes typical of neuronal and cardiac membrane under physiological conditions. One advantage of the methodology is that it offers paradigm-changing advances in both the automation of channel model discovery in the service of rational drug design, and in the ability to utilize physically relevant data to constrain both Hodgkin Huxley and Markov state ion channel model development. Ion channels models can help to understand the function of neuronal circuitry and cardiac tissue, and to uncover the functional defect involved in a disease related to ion channel dysfunction. The models developed by the method can be used to predict the mechanism of modulator action on ion channels. Rational drug design can be guided by this process, particularly when implemented in an automated fashion.

The details of one or more embodiments are set forth in the description below. Other features, objects, and advantages will be apparent from the description, and from the claims.

### DETAILED DESCRIPTION

The way in which Hodgkin and Huxley utilized electrophysiologically measurable data to constrain the parameter selection for their sodium and potassium ion conductance models has remained the paradigm for present work. Starting from a quiescent resting voltage, they applied a fixed voltage to membrane containing the voltage-gated conductances, and measured the membrane current flowing from the onset of the voltage change. This method, called the voltage clamp, allows the simplicity of studying the dynamic response of a channel, over time, at a single voltage. Its simplifying benefit is the avoidance of the inherently nonlinear complexity presented by voltage-gated currents and their effects on membrane under conditions of changing voltage. Thus a series of activation steps is applied from a quiescent fixed holding voltage, and the time course of channel current measured. To disentangle the process of inactivation, the shutting of the channel which occurs in some channel types, another simplifying protocol was used, whereby from rest an intermediate plateau of membrane voltage was applied, called a pre-pulse, followed by a voltage command to a highly conducting level where the activatable channel population remaining is revealed. The fixed voltage clamps applied to gain data for these models are quite unlike the voltage environment actually experienced by the membrane during physiological activity. In the case of neurons or cardiac channels, this is more typically spikes of brief very high voltage. It is rarely noted that the performance of real channels during realistic voltage histories is not revealed by HH model protocols and that channel dynamics elicited during trains of voltage spikes is simply not present in the data used for HH model development. Ion channel models described here can be used to model data recorded with a realistic voltage history. Unlike HH, the models are not limited to recreating experimental results where the simplifying holding voltage and pre-pulse procedures have been used.

Some deficiencies of HH models have long been noted. Soon after the nature of the conducting component of ion channels as integral membrane spanning single proteins was determined, it became clear that the separation of activation and inactivation processes inherent in the HH model format is unlikely to be biophysically accurate. Given that shifts in channel state arise from allosteric transitions between configurations of different potential energy in a single protein, it is unlikely that the shifts causing steps leading to activation do not change the potential energy of a subsequent allosteric shift leading to inactivation. Indeed, studies of the gating of inactivation of the sodium ion channel have demonstrated that it is in fact linked to activation, with evidence suggesting that the apparent voltage-dependence of inactivation characterized by Hodgkin and Huxley is actually derived from its linkage to the voltage-gated activation transition. So the processes of activation and inactivation are in fact intimately linked, and the HH formalism, which depends upon their absolute independence, is not an apt model of the biophysical channel transformations occurring in gating. Ion channel models described here can account for inactivation and the relationship between activation and inactivation, more faithfully modeling the behavior of real ion channels.

It is increasingly clear that there are functions of physiological importance which occur during trains of spikes but which are not apparent in HH voltage steps. They therefore cannot be and are not incorporated into HH models. One important example is a type of inactivation of the sodium ion channel known as cumulative inactivation. See Rudy, B., (1981) Journal of Physiology, 312:531-549, which is hereby incorporated by reference in its entirety. It differs from the sodium ion channel inactivation classically described in HH protocols in that it occurs similarly during either a brief or long step; it thus seems associated with the process of transition from closed to open. Once occurring, the recovery from this inactivated state is very slow. As a result, a train of brief depolarizations such as a train of spikes begins to progressively deplete the effectively available channel population. In view of the complexity of temporal neuronal membrane during real function, cumulative inactivation of sodium ion channels is likely to be a central aspect of their functional properties, yet it is not reflected at all in HH models of the sodium ion channel. The ion channel models described here can include the effects on ion channels of trains of spikes, including cumulative inactivation effects.

There is an alternative form of channel model that is more directly related to the biophysical structure of ion channels. A channel is envisioned as existing in one of a set of distinct relatively stable states, of differing potential energies. The probability of lying in each state has a temperature-sensitive kinetic rate of transition into any of the others governed by the potential energy barrier between the states as provided in Boltzmann/Eyring rate theory (Hille 2001). Thus the rate of change of the probability of the channel lying in each of its states is governed by a first order differential equation, with the rates functions of voltage, providing a straightforward means of predicting the probability of the channel existing in each of its states as a function of time. The potential energy between states can be made voltage dependent by positing a charged voltage sensor, of variable valence, which adds or subtracts to the potential energy barrier between two states as the membrane voltage changes. In analogy with the allosteric shifts of a single channel protein, in the Markov state (MS) class of models the states are all interlinked.

Markov state models are more powerful than HH models, which are subsumed in them as a subset where activation and inactivation transitions are all independent of each other. MS models include a much broader class where transitions between any states may occur. For example the form of cumulative, slowly recovering inactivation of the sodium ion channel entered during spikes, as mentioned above, is not well captured in the HH formalism, is readily captured in a MS model. In one such solution a transition state preceding the open state is a route to a slowly recovering inactivated state. As mentioned above, this secondary form of sodium ion channel inactivation may have ubiquitous damping effect, depleting the available sodium ion channels following repetitive activity, and simulations of neuronal circuitry must therefore incorporate it. While HH models are not equipped to do so, Markov state models are.

Markov state models of ion channels are defined by their state structure, and the voltage dependence of transition rates between all states. However, existing methods available for determination of the value of the parameters governing the voltage dependence of transition rates between each pair of states were limited, and cumbersome. The primary method was described by Colquhoun and Hawkes, and is referred to as Q-matrix theory. See Colquhoun D., and Hawkes A. G. (1981) Proceeding of the Royal Society of London B, 211:205-235, which is hereby incorporated by reference in its entirety. In brief, the steady state values of activation for fixed voltage clamp steps are used to infer transition rates between states by solution of the set of differential equations governing state dynamics, using inversion of a the matrix defining the differential equations governing transition rates. However, Q-matrix methods only apply to data gathered from a fixed voltage clamp step or steps. Solving for the transition rate parameters for a series of steps, as in the prepulse protocols used to assess inactivation properties, requires a sequence of laborious solutions for the set of piecewise fixed voltage steps, with initial probabilities of occupancy in each state of the MS model the terminating values of the prior step. Not only are the methods cumbersome even for the inference of model parameters from simple voltage clamp steps, but it is infeasible to utilize data from more complex voltage clamp applications. Thus data from physiological voltage histories, such as spikes, trains, of spikes, bursts, cannot be utilized in the development of MS models with conventional methods. As a result, important channel phenomena such as the cumulative slowly recovering inactivated state noted above may not be captured at all in Markov state models developed using conventional methods, a distinct limitation to their utility.

A method of using experimental data determines the structure and voltage dependence of transition rates for multistate ion channel models. The method is more powerful than those extant in that it is not restricted to use with data collected from simple fixed voltage clamp steps, but rather can be applied with equal ease to data collected with voltage commands of any complex, physiologically relevant form. Further, the method is largely automatic, a computer simulation-driven algorithm that can run without supervision, and takes full benefit from the expanding power of inexpensive parallel computation. The parameter space defining ion channel models is high dimensional. The interaction between changes in parameters, such as voltage sensor charges related to the transition between a particular pair of states, and the model channel's performance in comparison to a data trace, may be a complex function. The algorithm searches parameter space using the algorithms of evolution, a highly powerful means to automatically search high dimensional parameter spaces with complex relationships between fitness and parameter changes.

An evolutionary algorithm is a stochastic search method that mimics biological evolution. See, for example, Bäck, T., (1996) Evolutionary Algorithms in Theory and Practice, Oxford: New York, which is hereby incorporated by reference in its entirety. Members of a population are examined, and those having desired characteristics are said to be fit. Based on their fitness, certain members of the population are then selected and bred to provide a new generation of the population. Mutation and crossovers introduce changes into the new generation. The process can be repeated any number of times, until the members of the population attain a desired level of fitness. Aspects of selection, reproduction, mutation, crossover and fitness are discussed in more detail below.

In brief, the method starts with an encoding of a general ion channel model as a string of parameters. A first generation of a family of channel models is bred. Then each model channel in this first generation is used to simulate the set of voltage traces available in the experimental dataset, and a systematic comparison is computed between the current traces produced by each candidate model in this first generation and measured data. Thus the fitness of each of the first generation of model channels is calculated, by integrating the error between the model channel current trace and the measured traces in the dataset. The best performing (even if very bad) members of this first generation are retained, and are used to breed a second generation of channel models. To do so, the parameters of the retained channels are perturbed and/or exchanged, using evolutionary operators such as mutation, crossover and exchange to utilize the best performing channels of the past generation to breed the family of channels in the next. The foregoing process is then iterated, another set of somewhat better channel models is retained from the second generation, and the third is bred.

This process can be iterated for any number of generations, such as 50 to 500 generations, and with each the best model channel improves. The rate of improvement is fast initially, as the poor performance of the crudely chosen initial generation of candidate channel models is rapidly improved. As channel performance gets better, improvement in more incremental, but observation of this system suggests that there are still occasional abrupt jumps in fitness, reminiscent of punctuated equilibrium described in biological evolution.

This novel algorithm can be highly parallel in its computation, since the most of the floating point load occurs when each channel model in the generation is simulated using the voltage traces in the experimental data, and its fitness computed. Thus the computing power of distributed parallel computing systems, in which a large number of commodity PC's are coordinated by a central master, so-called Beowulf clusters can be ideally suited to be applied the methods for the evolution of channel models described here.

The described algorithm can be carried out by computer for an unlimited number of generations without supervision. Tests show a remarkably powerful convergence upon channel model solutions. Once the initial choices controlling the mode of evolution (i.e. the frequency and extent of allowed mutations, the manner of crossover or exchange, the policy for retention of the best of a past generation to seed the next, etc.) and the choice of data for fitness calculation (the set of experimental voltage-clamp traces to be applied to the simulated channels, and the weighting applied to them) are made, the evolution process proceeds to better and better candidate models with unsupervised computation. The critical attractiveness of a channel development algorithm that proceeds with unsupervised computation is that the enormous reduction in man-hours needed for the development of a channel model using this system, when compared with conventional methods, enables the systematic study not only of the enormous number of individual isolated channels for which voltage clamp records have become available, but of the effects of the myriad of modulators of channel function for each channel of interest. A modulator is a substance that alters the behavior of an ion channel, such as a ligand or inhibitor. An enzyme that chemically modifies an ion channel can also be a modulator, for instance a kinase or a phosphorylase.

The greatest advance inherent in the described method is not, however, simply its suitability to automatic unsupervised parallel computation. It is the immediate ability to use data collected during application of complex voltage clamp waveforms, much closer to the physiological voltage patterns experienced by channels in neuronal or cardiac membrane, as the constraint for model development. The present method, its power and suitability to automated execution invites the systematic prediction of the mechanisms underlying modulator action upon channels, by evolving a MS model of a control channel, of the same channel after application of modulatory substance, and systematically comparing the two to ascertain the changes effected. Rational drug design may be guided by the same process, wherein the success of candidate drugs in effecting a desired shift in channel function may be assayed systematically by assessing the shifts in the MS model of the channel evolved from voltage clamp currents measured after application, providing systematic guidance for rational drug design. Unlike extant methods for parameter determination for either HH or MS models, the algorithm described here allows use of voltage clamp data with voltage patterns of physiological complexity, without additional computational effort compared to unphysiological fixed voltage patterns. For example, a voltage pattern consisting of a train of brief 100 mV spikes is a very natural voltage environment in neuronal or cardiac membrane. This pattern can be, and has been, applied in experimental voltage clamp and the resulting currents flowing through channels of interest measured. While such data on the performance of channels in natural voltage environments have long been available, it has not yet been possible to use it to constrain channel models, because both classic HH models as well as extant methods for determining parameters of MS models depend upon analysis of responses during nonphysiological fixed steps of voltage, such as the activation and inactivation series.

In contrast to earlier methods, no more computational effort is required to calculate the response of a model channel to an arbitrarily complex voltage clamp history than to a flat voltage step. The difference between the resulting simulated current trace and the analogous measured trace, whether measured by RMS error or any other fitness measure, is again computed without additional effort whether the applied voltage clamp pattern is simple (for instance, flat) or complex (for instance, a replay of neuronal firing patterns). So the present algorithm opens up the novel possibility of using physiological voltage patterns to constrain development of ion channel models. The entry into the slowly recovering sodium ion channel state referenced above, which is overlooked in HH model development using classic activation and inactivation steps, becomes an effortlessly applied constraint during the development of a MS model of the sodium ion channel using the class of evolutionary algorithms described here.

The methodology for the evolution of ion channel model structure and transition rate parameterization is described below. A general ion channel model can be parameterized as a string of real numbers. In order to apply an evolutionary algorithm to ion channel models, a general ion channel model can be parameterized as a string of numbers. For example, for an MS model, this can be done as follows: the first three numbers of the string are the number of closed (N_{C}), open (N_{O}) and inactivated (N_{I}) stable states in which the channel may exist. For each pair of states, {S₁,S₂} the rate of transition from S₁ to S₂ (r_{S1,S2}) as a function of voltage V and temperature T may be expressed according to eq. 1.

r_{S1,S2}=A_{S1,S2}Te^{V-V}^{_{2S1,S2}}^{)/k}^{_{S1,S2}} (1)

Thus the parameters governing the rate of transition S₁→S₂ are a rate, A_{S1,S2}, a voltage equilibrium point, V_{2 S1,S2}, and a slope k_{S1,S2}. V2 is related to the energy barrier for the transition, and 1/k is proportional to the amount of charge on the voltage sensor associated with the closed state, and is linearly related to the role of temperature via the Boltzmann equation, as it is employed in Eyring rate theory (Hille 2001). There is symmetry in the expression for the rate of the reverse transition S₂→S₁ (eq. 2).

r_{S2,S1}=A_{S1,S2}Te^{-(V-V}^{_{2S1,S2}}^{)/k}^{_{S1,S2}} (2)

so that the number of parameters required to compute the instantaneous transition rates as functions of voltage between all stable states (N_{S}=N_{C}+N_{O}+N_{I}), is 3*N_{S}*(N_{S}−1)/2. In this, the number of distinct transitions is N_{S}*(N_{S}−1), with the factor 1/2 accounting for the symmetry between forward and backward transitions. For example, for a model channel incorporating 2 closed, 1 open, and 2 inactivated states, the number of kinetic parameters required to specify all transition rates as functions of voltage is 3*5*4/2=30. In practice, transition rates between some pairs of states may be very close to 0.0 for all physiological voltages, indicating that the two states are not effectively connected.

The final parameters required for the model are relative permeabilities to extracellular sodium, calcium and potassium ions, from which Nernst reversal potentials and their dependence upon external and internal ionic concentrations may be calculated. Assuming the driving force is largely set by these 3 ions, so P_{K+}+P_{Na+}+P_{Ca2+}=1.0, only the percentages of total permeability associated with sodium ion and calcium ion, P_{Na+} and P_{Ca2+} need be specified.

The full vector of real numbers associated with each channel, then, has the following appearance:

{N_{C}, N_{O}, N_{I}, V_{2 C1,C2}, k_{C1,C2}, A_{C1,C2}, V_{2 C1,C3}, k_{C1,C3}, A_{C1,C3} . . . P_{Na}, P_{Ca}.}

As described above, N_{C}, N_{O}, and N_{I} represent the number of closed, open and inactivated states in the model. Each set of three parameters following contains the information to define transition rates between two states. Here the first triplet of parameters (V_{2 C1,C2}, k_{C1,C2}, A_{C1,C2}) is for the transition between the first and second closed states, C1 and C2, respectively. The next triplet is for the transition between the first and third closed states, C1 and C3. Additional triplets of parameters, each listing V₂, k, and A for a pair of states, are added until all transitions have been described. The permeability parameters P_{Na+} and P_{Ca2+} are last.

When channel transition rates are governed by the concentration of calcium ion as well as (or instead of) voltage, the parameters specifying the transitions dependent upon calcium ion binding, typically a Hill coefficient and a dissociation constant, can be included. Calcium ion binding gated models will not be illustrated in the example provided here.

When implementing crossover operations, it can be convenient to break the vector up into the three integers specifying the state structure, followed by a real numbered vector of the triplets of parameters specifying the rates of transition as functions of voltage, and a final pair of real numbers specifying relative permeabilities. Thus, in an example model channel with 2 closed, 1 open, and 2 inactivated states, the vector of numbers specifying its function in full, including transition rates between all states as functions of voltage as well as reversal potential in any ionic media, consists of a 35 dimension vector, of which 30 are variables specifying transition rates as functions of voltage:

{2 closed states, 1 open state, 2 inactivated states}

{V_{2 C1,C2}, k_{C1,C2}, A_{C1,C2}, V_{2 C1,O}, k_{C1,O}, A_{C1,O}, V_{2 C1,I1}, k_{C1,I1, A}_{C1,I1}, V_{2 C1,I2}, k_{C1,I2}, A_{C1,I2}, V_{2 C2,O}, k_{C2,O}, A_{C2,O}, V_{2 C2,I1}, k_{C2,I1}, A_{C2,I2}, V_{2 C2,I2}, k_{C2,I2}, A_{C2,I2}, V_{2 O,I1}, k_{O,I1}, A_{O,I1}, V_{2 O,I2}, k_{O,I2}, A_{O,I2}, V_{2 I1,I2}, k_{I1,I2}, A_{I1,I2}}

{P_{Na}, P_{Ca}},

whereas the full specification of a simpler channel with 2 closed, 1 open and no inactivated states is a 14 dimension vector:

{2 closed states, 1 open state, 0 inactivated states}

{V_{2 C1,C2}, k_{C1,C2}, A_{C1,C2}, V_{2 C1,O}, k_{C1,O}, A_{C1,O}, V_{2 C2,O}, k_{C2,O}, A_{C2,O}}

{P_{Na}, P_{Ca}}.

If experimental data that provides information about the permeabilities of the channel is available, the last two parameters can be considered fixed, and the 3 state channel is fully specified by 9 variables.

In order to begin the first round of channel model evolution, an initial generation is needed. The members of the initial generation, or seeds, can be generated by quasi-randomly specifying one or more initial MS model parameter vectors (using the above form), drawing initial values for V₂, k and A from ranges considered physiologically plausible given the limitations on energy differences and voltage sensor charges. As an example of an initial MS model for an sodium ion channel, consider a four-state pure sodium ion channel with two closed, one open and one inactivated state. Given the 4 states there are 4×3/2=6 distinct transitions and thus 18 transition rate parameters along with the 2 permeability parameters, so that there are a total of 20 parameters in this case (see Table 1).

TABLE 1
Exemplary Four-State Sodium Ion Channel Parameterization

| | Corresponding values |
|---|---|
| Parameters | of parameters |
| {N_{C}, N_{O}, N_{I}} | {2, 1, 1} |
| {V_{2 C1,C2}, k_{C1,C2}, A_{C1,C2}, V_{2 C1,O}, k_{C1,O}, | {−40, 8, 0.1, −45, 9, 0.2, |
| A_{C1,O}, V_{2 C1,I}, k_{C1,I}, A_{C1,I}, V_{2 C2,O}, k_{C2,O}, | −35, 7, 0.05, −42, 10, 0.2, |
| A_{C2,O}, V_{2 C2,I}, k_{C2,I}, A_{C2,I}, V_{2 O,I}, k_{O,I}, A_{O,I}} | −55, 2, 0.2, −46, 5, 0.4} |
| {P_{Na+}, P_{Ca2+}} | {1.0, 0.0} |

The triplets providing the parameters for the voltage-sensitive rate of transition between states are underlined for grouping clarity. Since there are limits to the physiologically plausible energy difference between states and the intervening energy barriers, as well as to the valence of charge on the voltage sensor exposed to transmembrane voltage for any state, it is appropriate to establish absolute ranges of perturbation selected to ensure that energy differences and voltage sensor charge levels are specified within physiologically plausible ranges, within which the perturbations used to create the initial generation of channels may be randomly, normally or otherwise distributed.

One or more seeds are created, either chosen randomly or with some reference to the observed properties of the channel, such as, for example, presence or absence of inactivation, apparent V₂ of activation, etc. An initial population with which to start the evolution process may be generated by taking the seed channel or channels and making copies of with each parameter randomly distributed in turn. Thus if the seed channel has V_{2 C1,C2} the voltage equilibrium point for the transition between closed state C1 and C2=−50.0 mV, then in generating the remaining members of the first generation, V_{2 C1,C2} may be randomly chosen from a range of −50.0±20.0 mV, with a similar process applied to all 20 real number parameters of this 4 state channel. Each newly specified alternative model channel becomes another member of the initial generation.

If the space of channels of with 2 closed, 1 open and 1 inactivated state is viewed as a 20 dimensional parameter space, then the initial generation may be viewed as a cloud of channels, centered on the seed channel or channels. 100 candidate channels per generation can be an adequate number for reaching good solutions, but more may be needed. More channels per generation always allows more power in the exploration of parameter space during the evolutionary process. It is not a requirement that the best channel model has parameters lying within the cloud defined by the initial generation. The perturbations of parameters enabled by mutation during the evolutionary process allows the cloud of points to migrate through parameter space during the evolution process.

Given a generation of model channels and a set of experimental voltage clamp current records, the performance of each of the model channels in the generation is simulated and systematically compared with the measured records. Each experimental voltage clamp trace consists of a voltage waveform applied to the membrane and the measured current.

Typically each experimental trace commences at a resting, or holding, potential which has been maintained long enough that the channel is assumed to have reached equilibrium to ensure repeatable and stationary membrane initial conditions. To commence the simulation, the model channel must first be initialized—that is, the probability of occupancy in each of its closed, open, inactivated states must be calculated. An analytic solution for the equilibrium point of the first order kinetic system of equations can be obtained by setting the all the derivatives to 0 and solving the associated matrix inversion. Alternatively, the equilibrium distribution of state occupancy at the holding voltage can be computed by starting from a random starting configuration (where the probabilities of occupancy in each state sum to 1.0 and are all ≦1.0), and integrating forward. This random starting channel configuration, of course not at equilibrium at the resting voltage, will flow toward an equilibrium set of state occupancies. The resting voltage is simply applied, and the system can be integrated forward until changes in all state occupancies become acceptably small. These values, which must be ascertained individually for each model channel in the generation, are then used as the initial state for each simulated voltage clamp applied to test the model channel. Once initialized, the permeabilities of each channel to sodium, potassium, and calcium ions can be used to compute the reversal potential associated with the channel using the Nernst or GHK equation. The permeabilities can vary among the model channel population.

The string of parameters summarizing each channel model contains the information needed to compute the rate of transition from each state to all others, with the equation governing the change from state S₁ (eq. 3).

$$\frac{ⅆ {S}_{1}}{ⅆ t} = - ( {r}_{S1 , S2} + {r}_{S1 , S3} + … ) \cdot {S}_{1} + {r}_{S2 , S1} \cdot {S}_{2} + {r}_{S3 , S1} \cdot {S}_{3} + … \qquad ( 3 )$$

The values of r_{S1,S2} are determined according to eq. 1; S₁, S₂, S₃, and so on represent the probability of occupancy of that state.

Thus the multistate ion channel models follow first order kinetics, with in general all the rates functions of voltage (and in some cases internal calcium ion concentration). For example, in the case above where there are 2 closed, 1 open, and 2 inactivated states, the differential equation describing the first closed state, C₁, is shown in eq. 4:

$$\frac{ⅆ {C}_{1}}{ⅆ t} = - ( {r}_{C1 , C2} + {r}_{C1 , O} + {r}_{C1 , I1} + {r}_{C1 , I2} ) \cdot {C}_{1} + {r}_{C2 , C1} \cdot {C}_{2} + {r}_{O , C1} \cdot O + {r}_{I1 , C1} \cdot {I}_{1} + {r}_{I2 , C1} \cdot {I}_{2} \qquad ( 4 )$$

where r_{C1,C2} is the transition rate between the first and second closed states, according to eq. 1, and C₁, C₂, O, I₁ and I₂ are the occupancies of, respectively, the first and second closed states, the open state, and the first and second inactivated states.

Similar differential equations can be written for C₂, O and I₁, with the probability of occupancy in I₂=1−C₁−C₂O−I₁. The time derivative for each state is then numerically integrated forward to obtain the probability of occupancy in each state as a function of time. The way in which an simulated current trace is calculated from the probability of occupancy in each state as a function of time depends on how the experimental data was recorded.

The experimental data can include the current flowing through a population of (presumed identical) a large number of channels, as is the case in whole cell oocyte measurement for instance. To simulate such data, the current at each time point can be computed from the probability of the channel being in the open state or states, the ion permeability, and the instantaneous driving force. Only the open state can conduct ions, and the current will depend on both the proportion of channels in the open state and the ion permeability. Current is the product of the total proportion of channels in the open state, times the conductance density, times the driving force for the ion to which the channel is permeant.

The experimental data can instead include single channel measurements. In this case, the conducting state of the channel at each time interval is selected from the available states according to the probabilities of being in each state updated at each time interval. These can be averaged over a number of repeated trials corresponding to experimental procedure, and histograms of duration of open and closed intervals, and other statistics comparable to those collected during single channel experimental measurement, can be compiled.

A simulated current trace, produced upon application of the same voltage waveform as an experimentally measured voltage clamp current traces, can be compared to the experimental trace to provide a fitness. In each generation, the fitness of each channel model is computed and ranked or listed in order of fitness. The fitness measure can be a pointwise integration of error, or it can be a higher level signature of the trace such as its Fourier decomposition. The fitness can be a weighted integrated RMS error summed for a group of experimental voltage clamp trials. The fitness can be weighting to assign greater importance to regions of the experimental trace that reveal more information about channel dynamics. For example, in a long voltage trace the period immediately after application of a voltage command may be richer in information than a long static section of the experimental trace after the channel has equilibrated to the new voltage. More emphasis can be assigned to the dynamic section of the current trace by integrating RMS error with differential weighting assigned to a different section of the trace. The Monte Carlo simulation of single channel data can be subjected to the same statistical analyses used for experimental single channel measurement, and then compared systematically to compute the fitness of the single channel model. In the following, fitness is defined with regard to comparison of macroscopic current data with that predicted from the ion channel model.

Given an experimental voltage clamp dataset (a set of measured current traces associated with experimentally imposed voltage clamp waveforms), fitness can be calculated for each model channel compared to each trace in the dataset. However, the set of voltage clamp data may be quite large, consisting of series of trials of many protocols. An alternative is to first evolve an accurate fit to one protocol, such as the activation series. Once a fit generation of models has been evolved for that protocol, another voltage clamp series from the body of data, such as the inactivation prepulse series, may be imposed. This may be considered incremental evolution, in the sense that “environmental challenges” are introduced one at a time. The first data series can be retained while adding the second, so that the population of channels does not migrate into a niche tailored for the inactivation series only, but rather adapts to reproduce the inactivation series while maintaining performance on the activation series.

In summary, the procedure to apply the set of experimental voltage clamp series available is: 1) for each channel, for each voltage clamp trial, first initialize the resting state of the channel at the holding voltage used in that experimental trial; 2) using the experimental voltage waveform applied, integrate the channel forward, recalculating the voltage dependent channel transition rates whenever applied voltage changes; 3) for each trial, compute a fitness of the channel, such as the weighted RMS error for example, versus each experimental trial, summing this error for all traces in the experimental voltage clamp series; 4) rank channel models in the generation by fitness, select the surviving members, and use them to breed the next generation; and 5) iterate this procedure. Step 4, the evolutionary machinery, is described below.

When a generation of channels has been produced, and its performance on the series of voltage clamp trials simulated, and the fitness of each member of the generation computed as described above, the channels are then ranked based upon their fitness. Thereafter, a set of the best-performing channels are culled from this generation; this set will be used to generate, or breed, the next generation. The whole procedure will be iterated with the next generation.

There is a taxonomy describing the different choices as to which members of the present generation to keep (Bäck 1996). One choice is to simply keep the best performing 10% of the model channels (10 of 100) ion the next generation, and use them to generate the remaining 90% of the next generation. A common alternative choice of composition for the surviving set later used to breed the next generation is to keep C copies of the fittest N_{f} exemplars in proportion to their fitness. It may also be desirable to let survival be a stochastic process, whereby the probability of survival is proportional to fitness, but even unfit models may survive to contribute elements of their parameter to the next generation, in order to enrich the diversity of elements of solutions which may be incorporated through the breeding process into individuals of the next generation. The best members of the prior generation can optionally be required in the next generation thus ensuring that the fittest member of each generation is at least as fit as that of the prior. All these selection-related choices, along with those governing the evolutionary operators of mutation, crossover and exchange discussed below, may themselves be systematically varied to produce the most effective evolution processes. Thus the parameters of the evolution process itself may be evolved, a notion for which the term “meta-evolution” has been coined (Bäck 1996). Whichever choice is made as to exactly how the set of individuals retained from a generation in order to breed the population of channel in the next generation, hereafter we refer to that set of individuals as the surviving set.

Once the surviving set of a just-completed and ranked generation is culled, it remains to use it to breed the next generation. The evolutionary process allows a variety of means to take one, two or more members of the surviving set to breed new models. These perturbations or exchanges of the information contained in the string of parameters which together specify the channel model are to some degree inspired by those operative in biological genetic reproduction. However, there are recombination operations such as individual parameter exchange, wherein each element of the parameter string may be exchanged between two individuals, which are not physically feasible in biological evolution but are of course implementable in synthetic evolutionary process described here, and which may have more power than crossover, the closest biological counterpart.

Mutation involves the unilateral alteration of one or more parameters, within a specified range. It does not mix properties between individuals of the surviving set, but rather creates new individuals by perturbing the existing ones. A common approach is to apply a probability of mutation, generally in the range of 0.5 to 10%, to each parameter in turn. If a parameter is stochastically selected to be mutated, then its value may be changed by a random or normally distributed value, with the direction of change centered on 0.0 so that the perturbation from the prior value can be in either direction. The mutation operation is applied to each parameter in turn, and a new candidate model channel created thereby. This procedure may cycle through the members of the surviving generation in turn, until a full new generation is produced.

The range of perturbation, or mutation radius, can be set as a tight neighborhood around the existing value, or a wider range. As the fitness of a generation converges upon a high fitness populations of solutions to the channel model problem, the rate of mutations and the mutation radius can be contracted, since abrupt departures of parameter values are not likely to increase fitness in a highly evolved channel, with the more graded variations desirable in refining a highly evolved and already very fit channel model.

A commonly utilized form of exchange of parameters between two elements of the surviving set of channel models from the past generation is crossover. As with mutation, it exists in biological evolution, comprising the sexual process of deriving one surviving genetic string by joining complementary pieces of two parents. Pairs of model channels from the surviving set can be selected at random, or systematically to utilize all equally, and the location or locations of crossover along the parameter vector can be varied along the real valued string in a random fashion, or at breakpoints corresponding to transition rate triplets.

A more general, and perhaps more powerful form of sexual interchange of parameters between elements of the surviving generation than crossover is parameter exchange. In this, again two members of the surviving population are paired, and a probability of exchange (analogous to the probability of mutation referenced above) is applied to each parameter along the string in turn, so that exchange is stochastic with respect to each parameter. If a parameter is selected for exchange, then its value is replaced by that of the corresponding parameter in the chosen exchange partner. This process, which does not occur in nature, may be applied between different partners in turn for each parameter, or between all parameters between a single pair of partners. The members of the surviving set may then be paired with partners randomly chosen from, or systematically rotated through, the balance of the surviving set, with new model strings generated at each iteration, continuing until the desired number of new channels is created.

The order of application of mutation and exchange may be reversed, so that parameters of the surviving set are exchanged according to one of the methods described above to create the next generation, and thereafter the mutation operation applied to each member of the generation thereby created. The surviving set may itself be left intact, to ensure that the value of the fitness of the fittest model does not decline.

The procedure can be modified to allow discovery of the state structure of the multistate ion channel model. Competition can exist among alternative state structures. The foregoing procedures apply to the evolution of a set of transition rate and permeability parameters to optimize a multistate channel model, given a fixed number of closed, open, and inactivated states. A method is needed to allow the evolutionary process to explore alternative state structures in an automatic way, allowing differing state structures to compete.

One approach is to allow a mixed population of MS models, with many variations of models for each of a number of different state structures. If the population is viewed as consisting of many “subspecies” each with the same number of closed, open, and inactivated channels, then reproductive interaction (parameter exchange) may be simply restricted to occur within the group of individuals of the same subspecies. The reason for this restriction is that exchange of parameters or crossover of portions of parameter strings may not function when the state structure is different. For example, if species A has one closed state and species B has two closed states, the voltage-dependent rates connecting the single closed to open state in species A may be the half the rate of the transition from the first closed state to the second, and from the second closed to the open state in species B, since the observed rate of activation of the channel from a resting closed state must be the same in both. Thus an exchange of rate parameters between states with different structure may not be functional, in analogy to the inability of different species to interbreed in nature.

In an evolutionary strategy where subpopulations of different subspecies intermix among themselves, there still remains full fitness competition, wherein the fitness of all the candidate models in all the subspecies subpopulations are ranked together, and, as one example of the survival operation the fittest 10% are kept. If successful members of differing state structures remain in a surviving set, then the next generation will contain a cohort representing each surviving subspecies, with a population of this subspecies in the new generation proportional in number to the representation of the surviving subspecies in the surviving set. A variation on this procedure which differs mainly in the organization of the computation is to evolve a level of initial fitness in separate populations of each subspecies to a high preselected benchmark, and then combine the subpopulations and begin to pit them against each other.

The rate of convergence to fit solutions can be slower for the ultimately optimum model structure. In this circumstance, the early stages of competition could threaten to eliminate all early crude examples of what would ultimately be the best channel structure. It is therefore desirable to enforce the maintenance of some members of each subspecies within each cohort once an equivalent level of preset fitness has been reached.

The diversity of state structure can be explored with direct mutation of the number of available states—there can be a need for probabilistic survival of initially unfit product of mutations. If variation of state structure is allowed, the integer parameters establishing the number of closed, open and inactivated states may simply be subject to mutation, or exchange. An increase (or decrease) in the number of available states will have a corresponding effect on the transition parameter triplets required, with each new state requiring parameters for the transition to all the other preexisting states. If these are included with initial random values, it can be very unlikely that the individual arising from that mutation will be initially fit, even if the new state structure is the perfect one for the actual channel. To enable a meaningful incremental exploration of state structures implemented by mutations in the numbers of states, stochastic survival rules become vital to enable initially unfit individuals, new products of a mutation in state structure, to further evolve their parameters. The same issue arises in biological evolution, where mutations are often fatal, and where promising ones, which after some refinement and coordination of related features via further evolution lead eventually to advances, initially cause a reduction in fitness. In biological evolution, survival is a probabilistic function of fitness in the environment, allowing experiments a chance to further evolve and, if the fitness increases quickly, the mutation can gain a foothold.

Complexity cost can be imposed on complex state structures. Clearly, allowing a range of state structures to compete increases the power of the procedure to arrive at a channel model that matches any given dataset. On the other hand, we do not desire any more than the minimum complexity that captures the all the relevant dynamic behavior of the channel to a satisfactory degree of fitness. One motivation for simplicity is computational economy in the eventual use of the model channel, in for example neuronal or cardiac simulations. Therefore when exploration of alternative state structures is included, a fitness cost may be attached to the number of states utilized, to prompt the algorithm to seek automatically a parsimonious solution.

A prerequisite to understanding much of the machinery of life is a predictive capacity to model ion channels and their nonlinear interaction. An automated tool to develop such models from voltage clamp data will be of great utility to researchers in academic and pharmaceutical settings. Ion channel models can help describe the behavior of muscle tissue, such as the heart; or nervous tissue, such as the brain or spinal cord. The models can reveal can be used to understand how a modulator affects channel mechanism—for instance, whether a particular modulator operates by locking a channel in a closed state or an inactivated state, or by changing the barrier height to a transition between states. Ion channel modulators can belong to important classes of molecules, such as drugs or toxins. Understanding how a modulator exerts its influence on an ion channel can have medical consequences.

A number of embodiments have been described. Nevertheless, it will be understood that various modifications may be made without departing from the spirit and scope of the invention. Accordingly, other embodiments are within the scope of the following claims.

What is claimed is:

1. A multistate model for an ion channel comprising a plurality of states, each state capable of a transition to each other state, each transition being described by a plurality of parameters, wherein the parameters are adjusted via an evolutionary algorithm.

2. The model of claim 1, wherein each transition is a voltage-dependent transition.

3. The model of claim 1, comprising two or more states.

4. The model of claim 3, wherein the states include an open state, a closed state, and optionally one or more inactivated states.

5. The model of claim 3, comprising three or more states.

6. The model of claim 5, wherein the states include an open state, a closed state, and an inactivated state.

7. The model of claim 1, wherein each transition is described by at least three parameters.

8. The model of claim 7, wherein the parameters include a rate parameter, a voltage equilibrium parameter, and a charge parameter.

9. An method of modeling ion channel behavior comprising:
simulating a result with a multistate ion channel model having two or more states;
comparing the simulated result to an experimental result to provide a measure of fitness; and
altering the multistate ion channel model based on the measure of fitness.

10. The method of claim 9, wherein the multistate ion channel model includes a plurality of states, each state capable of a transition to each other state, each transition being described by a plurality of parameters.

11. The method of claim 10, wherein each transition is a voltage-dependent transition.

12. The method of claim 10, wherein the multistate ion channel model is a member of a population of multistate ion channel models.

13. The method of claim 12, further comprising selecting a member of the population after comparing each simulated result to an experimental result.

14. The method of claim 12, wherein simulating the result and comparing the simulated result to an experimental result are performed in parallel on each member of the population.

15. The method of claim 12, wherein the states include an open state, a closed state, and optionally one or more inactivated states.

16. The method of claim 12, wherein each transition is described by at least three parameters.

17. The method of claim 16, wherein the parameters include a rate parameter, a voltage equilibrium parameter, and a charge parameter.

18. The method of claim 12, wherein the method is iterated until the measure of fitness reaches a predetermined level of fitness.

19. The method of claim 12, wherein altering the multistate ion channel model includes swapping a parameter of a first member of the population with a parameter of a second member of the population.

20. The method of claim 12, wherein altering the multistate ion channel model includes altering the number of states.

21. The method of claim 12, wherein the experimental result includes an electrical measurement of a cell.

22. The method of claim 12, wherein the experimental result includes an electrical measurement of at least a portion of a cell membrane.

23. The method of claim 12, wherein comparing the simulated result to the experimental result to provide a measure of fitness includes ranking the members of the population by the measure of fitness.

24. The method of claim 12, wherein simulating a result includes predicting ion channel behavior in the presence of a modulator.

25. The method of claim 12, wherein the experimental result includes an electrical measurement of a cell measured in the presence of a modulator.

26. The method of claim 12, wherein the experimental result includes an electrical measurement of at least a portion of a cell membrane measured in the presence of a modulator.

27. The method of claim 10, wherein the multistate ion channel model is a model for a sodium channel, a potassium channel, a calcium channel, or a combination thereof.

28. A system for modeling ion channel behavior comprising:
a data input device configured to provide electrical recordings of a cell or a cell membrane;
a data analysis device electrically connected to the data input device; and
an output device electrically connected to the data analysis device.

29. The system of claim 28, wherein the data input device includes an electrode for recording an electrical signal of a cell or a cell membrane.

30. The system of claim 28, wherein the data input device includes a stored library of electrical signals recorded from a cell or a cell membrane.

31. The system of claim 28, wherein the stored library of electrical signals recorded from a cell membrane includes a signal recorded in the presence of an ion channel modulator.

32. The system of claim 28, wherein the library of electrical signals recorded from a cell membrane includes a signal recorded after a train of electrical pulses was applied to the cell membrane.

33. The system of claim 28, wherein the data analysis device computes a mathematical simulation of an electrical recording of a cell or a cell membrane.

34. The system of claim 33, wherein the data analysis device compares the mathematical simulation to an experimental recording of a cell or a cell membrane.
